(19) United States
(12) Patent Application Publication
(10) Pub. No.: US 2009/0214488 A1
(43) Pub. Date: Aug. 27, 2009

(54) METHODS AND COMPOSITIONS FOR TREATING BASEMENT MEMBRANE DISORDERS

(76) Inventor: Raghu Kalluri, Weston, MA (US)

Correspondence Address: CLARK & ELBING LLP, 101 FEDERAL STREET, BOSTON, MA, 02110, US

(21) Appl. No.: 12/093,236

(22) PCT Filed: Nov. 9, 2006
(86) PCT No.: PCT/US06/43766

§ 371 Date: Dec. 4, 2008

Related U.S. Application Data
(60) Provisional application No. 60/736,138, filed on Nov. 10, 2005.

(51) Int. Cl. A61K 35/12; A61K 35/14; A61K 31/7088

(52) U.S. Cl. 424/93.7; 424/529; 514/44 R

(57) ABSTRACT

The invention features methods and compositions for the treatment of basement membrane diseases and disorders, such as Alport's syndrome.

### FIELD OF THE INVENTION

This invention relates to methods and compositions for treating diseases and disorders associated with defects in intracellular and intertissue interactions by promoting the formation of functional basal lamina and extracellular membrane.

### BACKGROUND OF THE INVENTION

The basement membrane (BM), also referred to as the basal lamina, is an amorphous, sheet-like extracellular matrix of ˜50-100 nm in thickness. It is a form of the ubiquitous extracellular matrix (ECM) and is a basic component of all tissues. The BM provides for the compartmentalization of tissues, and acts as a filter for substances traveling between tissue compartments. Typically, the BM is found closely associated with an epithelium or endothelium in all tissues of an animal, including blood vessels and capillaries. Epithelial cell organization is determined by the BM, which acts as both a structural scaffold for cells in a tissue and a medium through which the cells communicate. In general, most cells are known to produce BM constituents. Some of the major components of the BM are type IV collagen, laminin, heparan sulfate proteoglycans, and entactin/nidogen. Minor components include agrin, SPARC/BM-40/osteopontin, fibulins, type VI collagen, type XV collagen, and type XVIII collagen. The BM components are secreted by cells and self assemble to form an intricate extracellular network. The formation of a biologically active BM is important to the development and differentiation of the associated cells, and for the development and function of tissues and organs, such as the kidney, lung, muscle, and intestine.

The importance of BMs was further realized when several genetic and acquired vascular diseases were associated with defects in BM components, such as type IV collagen. For example, Alport syndrome, which is an inherited disorder of the BMs of the kidney, eye, and ear, is caused by mutations in the α3, α4, or α5 chains of type IV collagen (COL4A3, COL4A4, and COL4A5). The protomeric form of type IV collagen is formed as a heterotrimer made up from a number of different subunit chains (α1-α6). Because the biological function of type IV collagen is critically related to the formation of an intact BM, mutations in the alpha chains of type IV collagen result in the generation of a dysfunctional BM. People who inherit defective genes for the “collagen” proteins in their BMs may develop progressive loss of renal function, deafness, and abnormalities of the eye.

In the kidneys, glomerular BMs normally act like filters, allowing fluid to move from blood vessels to urine while retaining protein and red blood cells within the bloodstream. Thus, one of the early signs of Alport syndrome may be leakage of small amounts of blood or protein into the urine during childhood. Collagen-containing membranes are also important for the shape of the lens of the eye and the structure of the inner ear.

The autosomal recessive form of Alport syndrome is caused when all copies of the COL4A3 gene are mutated in a person's cells. Most of the mutations identified in this gene cause a change in the sequence of amino acids in a region of the α3 chain of collagen type IV that is critical for combining with other type IV collagen chains. Other mutations decrease or prevent the production of the α3 chain of collagen type IV. Reduced amounts of these chains can negatively affect the production of the other type IV collagens that are also necessary for the proper formation of BMs. When this occurs, other kinds of collagen accumulate in kidney BMs, eventually leading to scarring of the kidneys and kidney failure.

Another disease associated with defects in the COL4A3 genes is thin basement membrane nephropathy, which is an autosomal dominant condition manifested by blood in the urine of the patient, but no other symptoms or signs of kidney disease; it is unusual for this condition to lead to kidney failure. This condition was often called benign familial hematuria.

Another BM disorder is epidermolysis bullosa (EB), which is a disorder characterized by painful blistering of the skin. Non-scarring forms may depend on the weather or temperature, and often appear on the hands and feet. The scarring varieties consist of open sores and blisters which can lead to disfigured, or mittened hands and feet, scar tissue in the mouth, or infection in the digestive organs. In the BM disorder, anchoring of the epidermis to the BM by the protein epiligrin is affected.

Another disorder involving dysfunction in BM formation is hereditary nephritis, which is an X-linked dominant disorder that involves a mutation in the α5 chain of collagen type IV (COL4A5). In most families with this disorder, thickening and thinning of the glomerular and tubular basement membranes occurs, with multilamination of the lamina densa in a focal or local distribution. Females usually are asymptomatic and have little functional impairment, whereas most males develop renal insufficiency between ages 20 and 30. Disease onset may resemble that of acute glomerulonephritis. The disease is detected by finding microscopic hematuria or recurrent episodes of gross hematuria. The urine may contain small amounts of protein and white blood cells. The nephrotic syndrome occurs rarely. There are no distinguishing histologic changes by light or immunofluorescence microscopy.

There is a need in the art for new and improved therapies for BM diseases and disorders.

### SUMMARY OF THE INVENTION

In a first aspect, the invention features a method for treating or preventing a basement membrane disease or disorder by administering to a patient in need thereof a stem cell from bone marrow, peripheral blood, or umbilical cord blood of a subject lacking the basement membrane disease or disorder, such that administering the stem cell treats or prevents the basement membrane disorder. In an embodiment, prior to administering the stem cell, a diagnostic test is performed on the patient to determine whether the patient has a basement membrane disorder. In another embodiment, the basement membrane disease or disorder is selected from Alport's syndrome, Knoblach syndrome, hematuria, epidemiolysis bullosa, thin basement membrane nephropathy, diabetic nephropathy, and hereditary nephritis, or any disease with defects in basement membranes and extracellular matrix. In a preferred embodiment, the stem cell is from bone marrow, peripheral blood (e.g., mobilized peripheral blood), or umbilical cord blood. In another embodiment, the stem cell is a mesenchymal stem cell or an endothelial stem cell.

In yet another embodiment of the invention, the basement membrane disease or disorder is characterized by a defect in one or more basement membrane components, such as collagen (e.g., type IV collagen, and in particular the α3, α4, or α5 chains of type IV collagen), laminin, a heparan sulfate proteoglycan, entactin/nidogen, agrin, SPARC/BM-40, osteopontin, and a fibulin.

Preferably, the stem cell is administered to the patient by injection into the patient's blood stream.

In a second aspect, the invention features a method for treating or preventing a basement membrane disease or disorder by administering to a patient in need thereof a composition that includes a stem cell that has been isolated from bone marrow, peripheral blood, or umbilical cord blood of a subject lacking the basement membrane disease or disorder. Administration of the composition treats or prevents the basement membrane disorder. In an embodiment, prior to administering the composition, a diagnostic test is performed on the patient to determine whether the patient has the basement membrane disorder.

In several embodiments of the first and second aspects of the invention, the basement disease or disorder is selected from Alport's syndrome, Knoblach syndrome, hematuria, epidermolysis bullosa, thin basement membrane nephropathy, diabetic nephropathy, and hereditary nephritis, or any disease with defects in basement membranes or extracellular matrix of a patient. The basement membrane basement membrane disease or disorder is characterized by a defect in one or more basement membrane components, such as collagen (e.g., type IV collagen, and in particular the α3, α4, or α5 chains of type IV collagen), laminin, a heparan sulfate proteoglycan, entactin/nidogen, agrin, SPARC/BM-40, osteopontin, and a fibulin. In another embodiment of the first and second aspects of the invention, the stem cell is from bone marrow, peripheral blood (e.g., mobilized peripheral blood), or umbilical cord blood. In another preferred embodiment of the first and second aspects of the invention, the stem cell is a mesenchymal stern cell or an endothelial stem cell. Preferably, the stem cell is administered to the patient by injection into the patient's blood stream. In another preferred embodiment, the patient is a human patient.

A third aspect of the invention features a method for treating or preventing a basement membrane disease or disorder by administering to a patient (e.g., a human patient) a vector (e.g., a cell, a viral vector, or a nucleic acid vector) that includes or encodes (e.g., if it is a nucleic acid vector) a functional basement membrane component, such that administration of the vector treats or prevents the basement membrane disorder. In a preferred embodiment, prior to administering the vector to the patient, a diagnostic test is performed to determine that the patient has the basement membrane disorder.

In an embodiment of the third aspect of the invention, the vector is a viral vector (e.g., an adenoviral vector) that includes a transgene in an expressible genetic construct, in which the transgene expresses basement membrane component that is absent or defective in the patient. In another embodiment, the vector is a nucleic acid vector, such as a plasmid, that is administered to the patient and that encodes a basement membrane protein that is absent or defective in the patient.

In yet another embodiment, the vector is a cell (e.g., an autologous or allogeneic cell, or a stem cell, such as an endothelial or mesenchymal stem cell) that has been modified to express the basement membrane component. In a preferred embodiment, the cell is an autologous or an allogeneic cell. In another preferred embodiment, the cell has been modified by infection with a viral vector (e.g., an adenoviral vector).

In several embodiments of the third aspect of the invention, the basement disease or disorder is selected from Alport's syndrome, Knoblach syndrome, hematuria, epidermolysis bullosa, thin basement membrane nephropathy, diabetic nephropathy, and hereditary nephritis, or any disease with defects in basement membranes or extracellular matrix of a patient. The basement membrane basement membrane disease or disorder is characterized by a defect in one or more basement membrane components, such as collagen (e.g., type IV collagen, and in particular the α3, α4, or α5 chains of type IV collagen), laminin, a heparan sulfate proteoglycan, entactin/nidogen, agrin, SPARC/BM-40, osteopontin, and a fibulin. In another embodiment of third aspect of the invention, the stem cell is from bone marrow, peripheral blood (e.g., mobilized peripheral blood), or umbilical cord blood. In another preferred embodiment of the third aspect of the invention, the stem cell is a mesenchymal stem cell or an endothelial stem cell. Preferably, the stem cell is administered to the patient by injection into the patient's blood stream. In another preferred embodiment, the patient is a human patient.

By “basement membrane disease or disorder” is meant a disease or disorder that results from a defect in the basement membrane (BM) or that affects the formation or function of the basement membrane (BM) or the extracellular membrane (ECM). A basement membrane disease or disorder can result from a defect in any component of the basement membrane, such as type IV collagen, laminin, heparan sulfate proteoglycans, entactin/nidogen, agrin, SPARC/BM-40/osteopontin, fibulins, type VI collagen, type XV collagen, or type XVIII collagen, or it can cause a defect to occur in a basement membrane component. A basement membrane disease or disorder is characterized by a dysfunction in the assembly of the BM or by a dysfunction in the development or function of tissues or organs, such as the kidney, liver, lungs, heart, skin, ovaries, testes, spleen, stomach, muscle, and intestines, associated with the BM. Non-limiting examples of basement membrane diseases or disorders are Alport's syndrome, Knoblach syndrome, hematuria, epidermolysis bullosa, thin basement membrane nephropathy, diabetic nephropathy, and hereditary nephritis.

By the term “engrafting” or “engraftment” is meant the persistence of cells of the invention in a particular location over time following transplantation of the cells into a mammal (e.g., a human).

By “heterologous gene” is meant that a nucleic acid molecule that encodes a protein and that originates from a foreign source or, if from the same source, is modified from its original form.

By “stem cell” or “pluripotent stem cell,” which can be used interchangeably, is meant a cell having the ability to give rise to two or more cell types of an organism and which is self-renewing. Examples of stem cells include totipotent stem cells, pluripotent stem cells, multipotent stem cells, and progenitor stem cells; these cells can be adult and embryonic stem cells. Mesenchymal stem cells (MSCs) are multi-potent cells that can replicate yet maintain their status as undifferentiated cells while possessing the potential to differentiate into specific mesenchymal tissues lineages, including bone, cartilage, fat, tendon, muscle and bone marrow stroma. As used herein, term “MSC” or “mesenchymal stem cells” of the invention comprises, without limitation, embryonic MSC, adult MSC or cord blood stem cells. Endothelial stem cells are multi-potent cells that can replicate yet maintain their status as undifferentiated cells while possessing the potential to differentiate into specific endothelial tissues lineages, including the tissues of the inside surfaces of body cavities, blood vessels, and lymph vessels.

Stem cells for use in the methods of the invention can be obtained from any source, e.g., bone marrow, peripheral blood, and umbilical cord blood. Cultured stem cells that can also used in the methods of the invention. Preferably, the stem cells are human stem cells.

By “transgene” is meant any piece of a nucleic acid molecule (for example, DNA) that is inserted by artifice into a cell transiently or permanently, and becomes part of the organism if integrated into the genome or maintained extrachromosomally. Such a transgene may include a gene that is partly or entirely heterologous (foreign) to the transgenic organism, or may represent a gene homologous to an endogenous gene of the organism.

By “transgenic cell” is meant a cell containing a transgene. For example, a cell transformed with an expression vector operably linked to a heterologous nucleic acid molecule can be used to produce a population of cells having altered phenotypic characteristics. A cell derived from a transgenic organism is also a transgenic cell so long as the cells contain the transgene.

By “transplant” or “transplanting” is meant administering one or more cells (or parts thereof), cell products, tissue, or cell culture products derived from cells that are grafted into a human host. Specifically, a transplant is produced by manipulating the cells described herein. These cells can be further manipulated to include heterologous genetic material, such as a transgene.

By “treating” or “treatment” is meant administering a pharmaceutical composition, e.g., one or more cells of the invention, or progeny or derivatives thereof, for prophylactic and/or therapeutic purposes. To “prevent disease” refers to prophylactic treatment of a patient who is not yet ill, but who is susceptible to, or otherwise at risk of, a particular disease. To “treat disease” or use for “therapeutic treatment” refers to administering one or more cells of the invention, or progeny or derivatives thereof, for the treatment of a patient already suffering from a disease to ameliorate the disease and improve the patient's condition. Thus, in the claims and embodiments, treating is the administration to a mammal either for therapeutic or prophylactic purposes one or more cells of the invention, or progeny or derivatives thereof.

By “vector” or “expression vector” is meant an expression system, a nucleic acid-based vehicle, a nucleic acid molecule adapted for nucleic acid delivery, or an autonomously self-replicating circular DNA (e.g., a plasmid). When a vector is maintained in a host cell, the vector can either be stably replicated by the cells during mitosis as an autonomous structure, incorporated within the genome of the host cell, or maintained in the host cell's nucleus or cytoplasm.

Other features and advantages of the invention will be apparent from the following Detailed Description, the drawings, and the claims.

### BRIEF DESCRIPTION OF THE DRAWINGS

FIG. 1A is schematic representation of the structure and the different cell types of the glomeruli. FIG. 1B shows photomicrographs of glomerular basement membrane sections from (i) a wild-type mouse, (ii) an α3 knock out mouse, (iii) a wild-type mouse following injection of bone marrow cells containing from a ROSA mouse, and (iv) an α3 knock out mouse following injection of bone marrow cells containing from a wild-type mouse. Panel (iv) shows significant restoration of the architecture of the glomerular basement membrane in the α3 knock out mouse following injection of ROSA cells.

FIG. 2A is a graph showing a time course of urinary excretion in COL4A3^{-/-} mice.

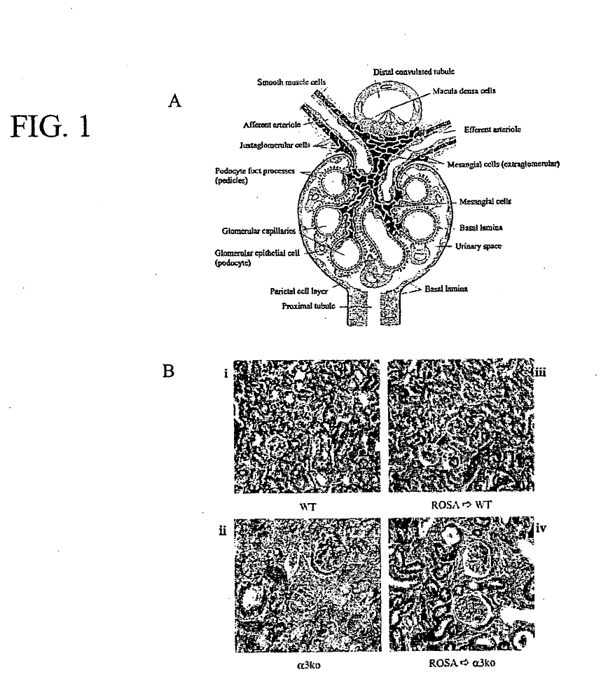

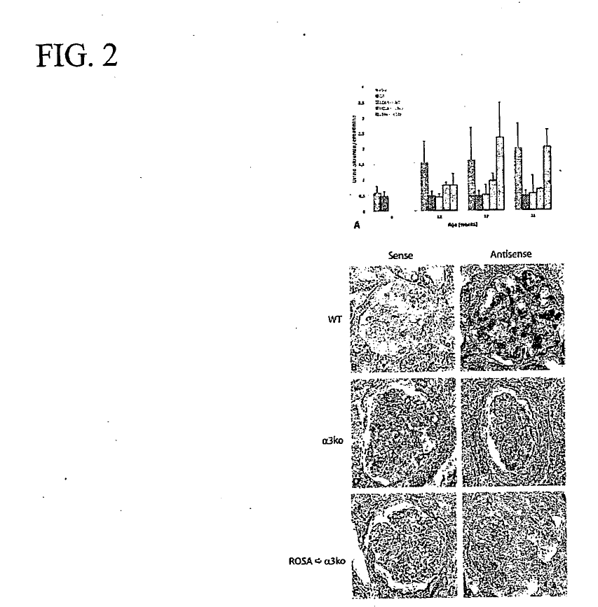

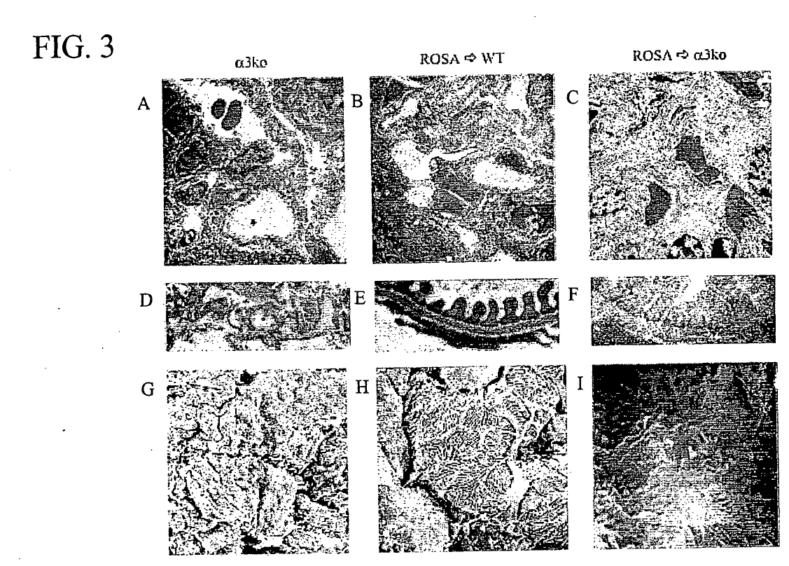

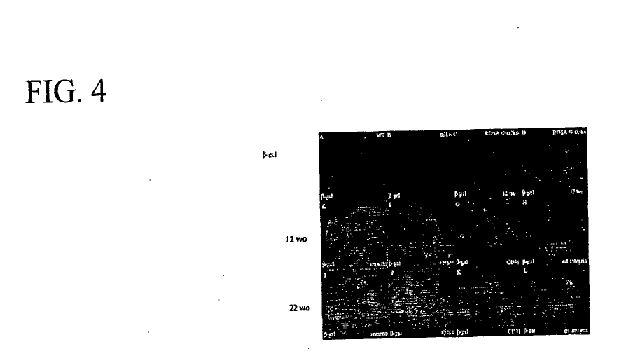

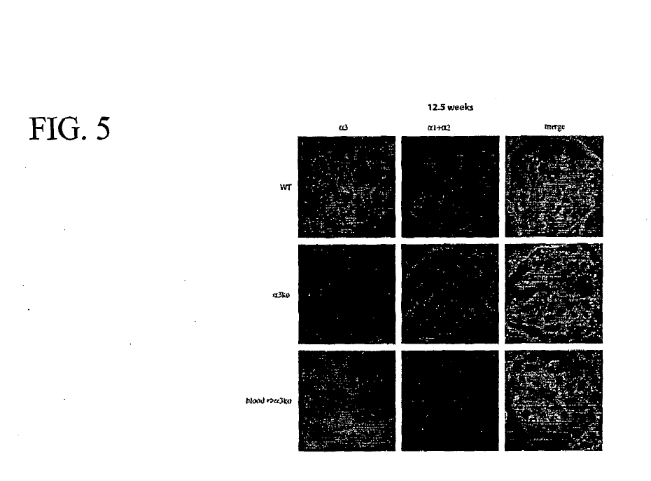

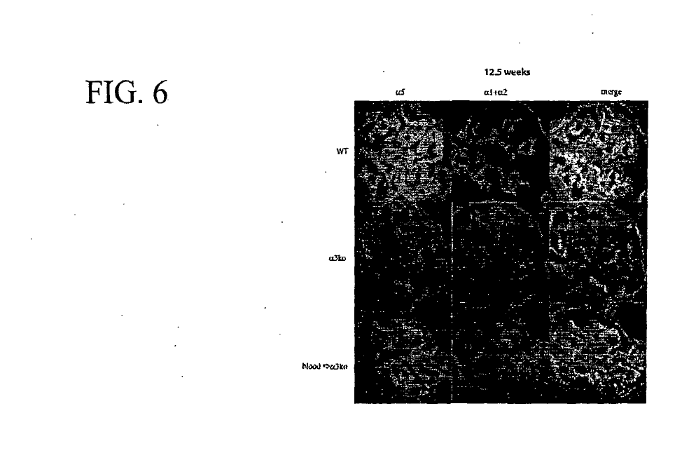

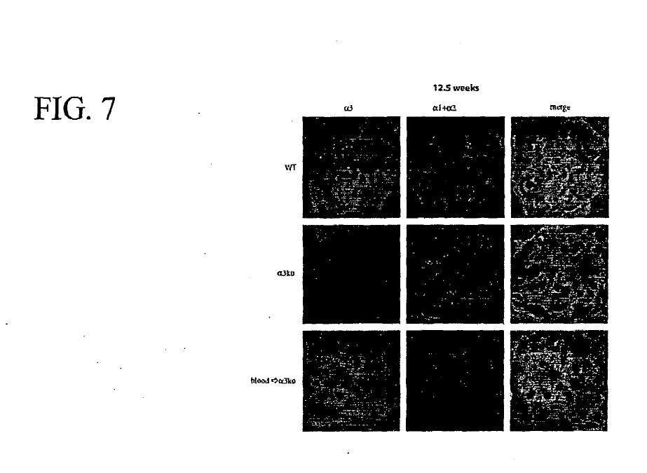

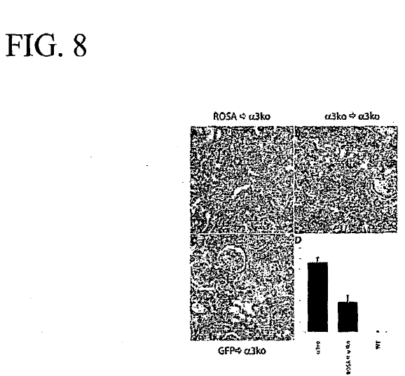

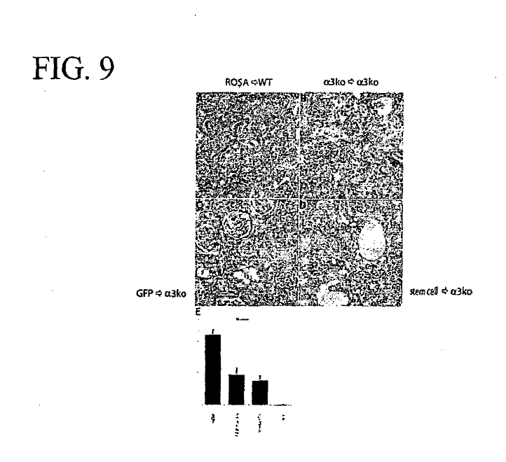

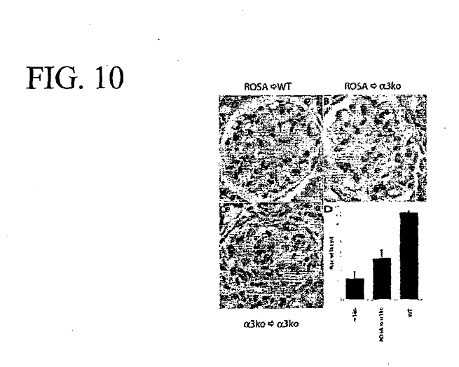

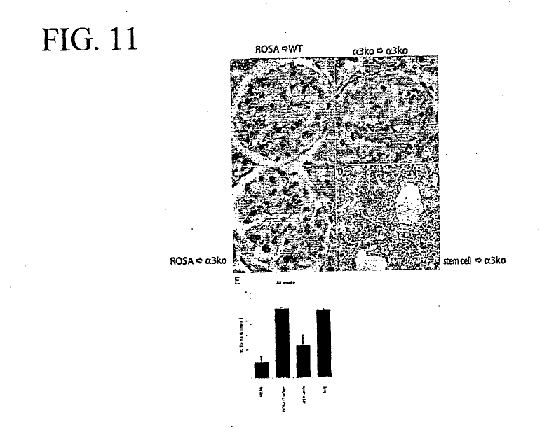

The bar graphs show urine albumin excretion at 8, 12, 17, and 21 weeks in COL4A3^{-/-} mice (red column), wild type mice (blue columns), wild type mice transplanted with ROSA cells (yellow columns), COL4A3^{-/-} mice transplanted with ROSA cells (green columns), and COL4A3^{-/-} mice transplanted with COL4A3^{-/-} cells (magenta columns). FIG. 2A indicates that untreated COL4A3^{-/-} mice and COL4A3^{-/-} mice administered COL4A3^{-/-} cells exhibit high levels of urine albumin excretion, whereas COL4A3^{-/-} mice administered ROSA cells exhibit reduced urine albumin excretion that more closely resembles that of wild type mice. The results are shown as the mean±SE. and indicate p<0.05 and p<0.01, respectively, compared with wild type mice. † indicates p<0.05, compared with COL4A3^{-/-} mice.

FIGS. 2B-2G are photomicrographs of glomerular basement membrane sections detecting type IV collagen α3 chain nucleic acid using sense and antisense probes in a wild type mouse, a COL4A3^{-/-} mouse, and a COL4A3^{-/-} mouse administered ROSA cells. FIG. 2B indicates that stem cells administered to a COL4A3^{-/-} mouse engraft in the kidney and initiate production of the α3 chain of type IV collagen, the gene missing in the kidney glomerulus of the COL4A3^{-/-} knock out mice. In wild type glomeruli, α3 chain mRNA of type IV collagen was expressed in podocytes; α3 chain mRNA expression was not detected in glomeruli of COL4A3^{-/-} mice. However, its mRNA began to emerge in COL4A3^{-/-} mice transplanted with bone marrow from wild type mice.

FIGS. 3A-F are photomicrographs of kidney sections from a COL4A3^{-/-} knock out mouse (A, D, G), a wild type mouse administered stem cells from the bone marrow of ROSA mice (B, E, H), and COL4A3^{-/-} knock out mouse administered stem cells from the bone marrow of ROSA mice (C, F, I). FIGS. 3A, 3C, and 3G show splitting, a basket-weave appearance, and lamination and thinning of the glomerular capillary basement membranes in COL4A3^{-/-} mice. Podocytes show microvillous transformation and foot process effacement. FIGS. 3B, 3E, and 3H show the normal phenotype of glomerular capillary basement membranes in wild type mice. FIGS. 3C, 3F, and 3I show that a bone marrow transplant from wild type Rosa mice inhibited foot process effacement and improved the appearance of glomerular capillary basement membrane; most notably the complex interdigitations of foot processes between the different podocytes could be seen. In COL4A3^{-/-} mice transplanted with COL4A3^{-/-} bone marrow, the interdigitation of foot processes of podocytes disappeared and formed a sheet-like structure.

FIGS. 4A-L are photomicrographs of glomerular capillary basement membrane sections from WT mice, COL4A3^{-/-} mice, and COL4A3^{-/-} mice administered bone marrow cells from ROSA mice showing that stem cells administered to COL4A3^{-/-} mice can engraft in the kidney and become glomerular endothelial, mesangial and podocytes within the diseased kidney. FIG. 4A is a photomicrograph of a glomerular capillary basement membrane section from an untreated wild type mouse that has been stained with β-gal (negative control; P-gal detects ROSA cells). FIG. 4B is a photomicrograph of a glomerular capillary basement membrane section from an untreated COL4A3^{-/-} mouse that has been stained with β-gal, also a negative control. FIGS. 4C and 4D are photomicrographs of glomerular capillary basement membrane sections from COL4A3^{-/-} mice administered wild type ROSA stem cells. FIGS. 4C and 4D shows that the ROSA cells engraft in the diseased kidney. FIGS. 4E-4H are photomicrographs of glomerular capillary basement membrane sections from 12 week-old COL4A3^{-/-} mice administered wild type ROSA stem cells. The sections are double-stained with β-gal (to detect ROSA cells) and entactin, synpo, CD31, and α1 integrin, respectively, which show that the ROSA stem cells become glomerular endothelial, mesangial and podocytes within the diseased kidney. FIGS. 4I-4L are photomicrographs of glomerular capillary basement membrane sections from 22 week-old COL4A3^{-/-} mice administered wild type ROSA stem cells. The sections are double-stained with β-gal (to detect ROSA cells) and entactin, synpo, CD31, and α1 integrin, respectively.

FIG. 5 shows a series of photomicrographs of glomerular capillary basement membrane sections from 12.5 week old WT mice, COL4A3^{-/-} mice, and COL4A3^{-/-} mice administered peripheral blood cells from a wild type mouse. The sections have been stained with antibodies that detect α3 or α1 and α2. The last panel shows the merge of the α3 and α1 and α2 panels. FIG. 5 confirms that COL4A3^{-/-} mice lack expression of the α3 chain of type IV collagen, but that administration of peripheral blood from a wild type mouse restores α3 expression in COL4A3^{-/-} mice.

FIGS. 6 and 7 are the same as FIG. 5, but showing a different series of photomicrographs of glomerular capillary basement membrane sections.

FIGS. 8A-8C are photomicrographs showing glomerular capillary basement membrane sections from COL4A3^{-/-} mice administered embryonic stem cells from a wild type ROSA mouse, embryonic stem cells from a COL4A3^{-/-} mouse, and embryonic stem cells from a wild type mouse modified to express GFP. FIG. 8D indicates that treatment of COL4A3^{-/-} mice with embryonic stem cells from wild type mice inhibits progression of tubular damage and interstitial fibrosis in the kidney glomerulus. These results are similar to that observed upon administration of wild type bone marrow cells to COL4A3^{-/-} mice.

FIGS. 9A-9D are photomicrographs showing glomerular capillary basement membrane sections from WT mice administered embryonic stem cells from ROSA mice (FIG. 9A), COL4A3^{-/-} mice administered embryonic stem cells from COL4A3^{-/-} mice (FIG. 9B), COL4A3^{-/-} mice administered embryonic stem cells from wild type GFP-expressing mice (FIG. 9C), and COL4A3^{-/-} mice administered embryonic stem cells from wild type mice (FIG. 9D). FIG. 9E is a graph showing that the administration of embryonic stem cells can attenuate kidney disease and decrease proteinuria.

FIGS. 10A-10C are photomicrographs showing glomerular capillary basement membrane sections from WT mice administered peripheral blood stem cells from ROSA mice (FIG. 10A), COL4A3^{-/-} mice administered peripheral blood stem cells from ROSA mice (FIG. 10B), and COL4A3^{-/-} mice administered peripheral blood stem cells from COL4A3^{-/-} mice (FIG. 10C). FIG. 10D is a graph showing that the administration of embryonic stem cells can attenuate kidney disease and decrease proteinuria.

FIGS. 11A-11D are photomicrographs showing glomerular capillary basement membrane sections from WT mice administered peripheral blood cells from ROSA mice (FIG. 11A), COL4A3^{-/-} mice administered peripheral blood cells from COL4A3^{-/-} mice (FIG. 11B), COL4A3^{-/-} mice administered peripheral blood cells from ROSA mice (FIG. 11C), and COL4A3^{-/-} mice administered embryonic stem cells from wild type mice (FIG. 11D). FIG. 11E is a graph showing that the administration of peripheral blood cells can attenuate kidney disease and decrease proteinuria.

### DETAILED DESCRIPTION

Applicant has determined that progenitor mesenchymal and endothelial stem cells, e.g., stem cells derived from bone marrow, peripheral blood, or umbilical cord blood, can be administered to a patient suffering from a basement membrane disease or disorder, e.g., Alport's syndrome, to treat or prevent the disease or disorder in the patient. The basement membrane disease or disorder causes Or results in the formation of dysfunctional basement membrane structures in the tissues of the patient. In Alport's syndrome patients the dysfunction in basement membrane formation is caused by a defect in the α3 chain of type IV collagen. Stem cells administered to the patient are recruited to, and engraft in, the patient's diseased tissue. Recruitment of the stem cells restores (at least partially) the expression of the α3 chain of type IV collagen in Alport syndrome patients, and promotes the formation of functional basement membrane. Applicant's discovery is applicable to the treatment and prevention of basement membrane diseases and disorders that are caused by defects in any basement membrane component, as well as other conditions that cause or are caused by defects in basement membrane proteins.

Accordingly, the invention features methods and compositions for treating or preventing diseases or disorders caused by or related to dysfunction in the formation of basement membranes (BM) or extracellular membrane (ECM). Non-limiting examples of diseases or disorders in which dysfunction in the formation of BM or ECM occurs include Alport's syndrome, Knoblach syndrome, hematuria, epidermolysis bullosa, thin basement membrane nephropathy, diabetic nephropathy, and hereditary nephritis. Treatment or prevention of BM diseases or disorders, according to the invention, involves correcting or mitigating the effects of the BM disease or disorder, e.g., by providing cells or tissues that express functional basement membrane components, such as collagen type IV (COL4), laminin, heparan sulfate proteoglycans, and entactin/nidogen. The cells or tissues can be autologous or allogeneic. In a preferred embodiment, the cells include stem cells. It is also preferred that the stem cells are capable of differentiating into endothelial or epithelial cells which can express and generate a functional BM.

Also included in the present invention is a method of treating or preventing BM diseases or disorders by administering a homologous or heterologous gene, or vector encoding the same, in which the homologous or heterologous gene encodes a functional BM component. The homologous or heterologous gene can be introduced into one or more cells of the patient, which are then capable of expressing functional BM components, thereby restoring the patient's ability to form functional BM and treating or preventing the BM disease or disorder.

### Treatment of BM Diseases or Disorders

The method of the invention can be used to treat any human patients, whether children or adults, who suffer from a BM disease or disorder. It is envisioned that cells or tissues expressing functional BM components are administered to the patient for the treatment of the BM disease or disorder. Preferably, the cells are typed for the patient using the standard six transplantation markers. It is preferable that the cells exhibit a 6/6 match, although cells exhibiting a 5/6 or a 4/6 match can be with any rejection that does occur being offset by using the standard methods described below, e.g., the administration of immunosuppressive agents, such as cyclosporin A or FK506.

In addition, the administered cells or tissue may be natural (i.e., unmodified by introduction of a genetic element), or they may be engineered to include one or more BM components that are defective in the patient (e.g., the patient's own cells or tissue may be modified to express functional BM components which replace the non-functional or defective BM components naturally expressed by the patient's cells and tissue).

### Cells for Transplantation

Cells or tissue for transplantation according to the methods of the invention can be isolated from a fetal, neonate, or adult source, including, e.g., bone marrow, embryonic yolk sac, fetal liver, fetal and adult spleen, blood, including peripheral blood (e.g., mobilized peripheral blood) and umbilical cord blood, muscle, brain, skin, kidney, liver, lung, heart, connective tissue, vascular tissue, and the like. If the source of the cells or tissue is bone marrow, the cells or tissue may be obtained from, e.g., iliac crests, tibiae, femora, spine, ribs, or other bone cavities. For isolation of bone marrow from fetal bone or other bone source, an appropriate solution, e.g., a balanced salt solution, may be used to flush the bone. Convenient buffers include Hepes, phosphate buffers, or lactate buffers. Alternatively, bone marrow may be aspirated from the bone in accordance with conventional methodology. In addition, the cells or tissues can be autologous or allogeneic.

In an embodiment, the cells or tissue are originally derived from the patient to which they are to be administered, i.e., the transplant is autologous, and the cells or tissue is modified to express one or more functional BM components that are defective in the patient.

In another embodiment of the present invention, bone marrow, umbilical cord blood, or mobilized peripheral blood is administered to a patient suffering from a BM disease or disorder for the treatment or prevention of that disease or disorder. In this embodiment, the bone marrow, umbilical cord blood, or mobilized peripheral blood is obtained from a donor and is administered to the patient without additional manipulation (i.e., the separation or purification of the cells or tissue). Alternatively, bone marrow, umbilical cord blood, or mobilized peripheral blood can be fractionated to separate various cellular components, with only a portion of the separated cellular components being administered to the patient. It is also possible to separate one or more cell types present in bone marrow, umbilical cord blood, or mobilized peripheral blood, such as stem cells, and to administer those cells to the patient; the stem cells may be administered in combination with other cell types, expanded prior to administration, genetically modified to express a BM component, or differentiated into a particular cell type prior to administration, such as a BM-producing cell. Stem cells, e.g., hematopoietic stem cells (HSCs), are commonly transplanted between individuals of a single species (and even from an individual to a cell culture system and back to the same individual); such cells have the advantage that they are amenable to nucleic acid transfection while in culture, and are, therefore, well suited for use in the invention. The cells administered to the patient exhibit the ability to engraft in the patient and to supply BM components within the body of the patient sufficient to treat or prevent the BM disease or disorder.

Stem cells for use in the invention are not limited to hematopoietic stem cells (HSC; e.g., LTR-HSCs), but also include stem cells of endothelial tissues, epithelial tissues, such as the skin and the lining of the gut, embryonic heart muscle cells, liver stem cells, kidney stein cells, and neural stem cells (Stemple and Anderson, Cell 71:973-985, 1992). The stem cells can be expanded under cell growth conditions, i.e., conditions that promote proliferation (“mitotic activity”) of the cells. HSCs useful in the methods of the invention are described in, for example, U.S. Pat. Nos. 5,763,197, 5,750,397, 5,716,827, 5,194,108, 5,061,620, and 4,714,680.

Cells for use in the methods of the invention can be obtained directly from tissues of an individual suffering from a BM disease or disorder, or from cell lines or by production in vitro from less differentiated precursor cells, e.g., stem or progenitor cells. An example of obtaining precursor cells from less differentiated cells is described in Gilbert, 1991, Developmental Biology, 3rd Edition, Sinauer Associates, Inc., Sunderland Mass. The precursor cells can be from any animal, e.g., mammalian, desirably human, and can be of primary tissue, cell lines, etc. The precursor cells can be of ectodermal, mesodermal or endodermal origin. Any precursor cells that can be obtained and maintained in vitro can potentially be used in accordance with the present invention.

Cells for use in the methods of the invention, such as stem cells, can be transfected with a plasmid comprising an operator sequence and a gene of interest and the transfected cells administered to a recipient mammal in need of the product of this gene. Transfection of stem cells, for example, is described in Mannion-Henderson et al., 1995, Exp. Hematol., 23: 1628; Schiffmann et al., 1995, Blood, 86: 1218; Williams, 1990, Bone Marrow Transplant, 5: 141; Boggs, 1990, Int. J. Cell Cloning, 8: 80; Martensson et al., 1987, Eur. J. Immunol., 17: 1499; Okabe et al., 1992, Eur. J. Immunol., 22: 37-43; and Banerji et al., 1983, Cell, 33: 729. These methods can be used to transfect many different cell types, and such methods may advantageously be used according to the present invention. Administration of transfected cells proceeds according to methods established for that of non-transfected cells.

The transplantation of cells, such as in a bone marrow transplant, is commonly performed in the art by procedures such as those described by Thomas et al. (New England J. Med., 292: 832-843, 1975) and modifications thereof. Such a procedure is briefly summarized: In the case of a syngeneic graft, no immunosuppressive pre-treatment regiment is required; however, in cases in which cells of a non-self donor are to be administered to a patient with a responsive immune system, an immunosuppressive drug must be administered, e.g. cyclophosphamide (50 mg/kg body weight on each of four days, with the last does followed 36 hours later by the transplant). Following pre-treatment, bone marrow cells, for example, (which population comprises a small number of stem cells), are administered via injection, after which point they colonize in the recipient host. Success of the graft is measured by monitoring the appearance of the administered cells in and around the organs and tissues of the patient by using immunological and molecular methods which are well known in the art, and by the production of functional BM associated with the organs and tissues of the patient due to engrafting of the administered cells. It is advantageous to optimize the rate at which engrafting of the administered cells occurs in the patient; therefore, a transplanted bone marrow sample comprising 10 to 100, or even 100 to 1000 stem cells should be administered in order to be therapeutically effective.

The use of stem cells in the treatment of human diseases has been shown to have efficacy. During the last few years several reports have demonstrated the ability of bone marrow to cross lineage boundaries and to implant and differentiate into different tissues including the kidney (Bailey et al., 2004; Krause et al., 2001; Ricardo and Deane, 2005). Ferrari et al. could demonstate that in immunodeficient mice after bone marrow transplantation marrow-derived cells migrate into areas of induced muscle degeneration, show myogenic differentiation, and participate in the regeneration of damaged fibers (Ferrari et al., 1998).

### Methods for Isolating Cells or Tissue for Transplantation

Methods for isolating the cells of the invention include positive or negative selection or a combination of both techniques. These two techniques, used alone or in combination, allow unwanted cells to be removed from the system and target cells to be harvested whenever desired. Procedures known in the art include, e.g., magnetic separation, using antibody-coated magnetic beads, affinity chromatography, cytotoxic agents joined to a monoclonal antibody or used in conjunction with a monoclonal antibody, e.g., complement and cytotoxins, and “panning” with an antibody attached to a solid matrix, e.g., plate, or other convenient technique. Techniques providing accurate separation include fluorescence activated cell sorters, which can have varying degrees of sophistication, e.g., using a plurality of color channels, low angle, and obtuse light scattering detecting channels, impedance channels, etc. A description of positive and negative selection techniques can be found in, for example, U.S. Pat. Nos. 5,925,567, 6,338,942, 6,103,522, 6,117,985, 6,127,135, 6,200,606, 6,342,344, 6,008,040, 5,877,299, 5,814,440, 5,763,266, and 5,677,136.

Optionally, the cells to be administered to the patient can be expanded using growth factors and cytokines, such as Flt3, SCF, VEGF, FGF, EGF, IL-3, IL-6, IL-11, and IL-12 may be added. If the cells to be expanded are stem cells, various in vitro and in vivo tests known to the art may be employed to ensure that these cells retain their pluripotentiality.

When desired, cell preparations having greater than 95%, usually greater than 98% of stem cells can be achieved using the methods described below for isolating, purifying, and culture expanding the cells.

### Transplantation

Methods for introducing cells for transplantation include but are not limited to intradermal, intramuscular, intraperitoneal, intravenous, subcutaneous, intranasal, and epidural routes. The cell-containing compositions of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Systemic Infusions

The cells can be administered by infusion into the patient by, e.g., intracoronary infusion, retrograde venous infusion (see, e.g., Perin and Silva, Curr. Opin. Hematol. 11:399-403, 2004), and intraventricular infusion. It is anticipated that human therapy is likely to require at least one infusion, and likely two or more infusions of any cell composition administered to treat or prevent a BM disease or disorder. Several infusions of cells can be administered over time, e.g., one on day one, a second on day five, and a third on day ten. After the initial ten day period, there can be a period of time, e.g., two weeks to 6 months without cell administration, after which time the ten-day administration protocol can be repeated.

Whether administered as a single infusion therapy or multiple infusion therapies, it is likely that the recipient will require immunosuppression. The protocols followed for this will follow the precedents now used in human transplantation for bone marrow replacement (i.e., cell transplantation), with such agents as cyclosporin A and FK506.

Direct Injection

Another possible administration route for the cells is via direct surgical injection (e.g., intramyocardial or transendocardial injection, intracranial, intracerebral, intracisternal injection, intramuscular injection, intrahepatic injection, intrarenal injection, and intrapancreatic injection) into the tissue or region of the body to be treated (e.g., the brain, muscle, heart, liver, pancreas, kidney, and vasculature, and tissues surrounding the organs in the body). This method of administration may also require multiple injections with treatment interruption intervals lasting from 2 weeks to 6 months, or as otherwise determined by the attending physician.

Implantation

The cells or tissue can also be administered by implantation into a patient at the site of BM disease or disorder or at a site that will facilitate treatment of the BM disease or disorder.

The amount of cells or tissue to be administered depends on patient needs, on the desired effect, and on the chosen route of administration. The cells or tissue may be administered by themselves, or they may be administered in conjunction with one or more biologically active agents to speed engraftment, e.g., an immune suppressing agent or an antibiotic.

### Gene Therapy

The cells or tissue may also be used for gene therapy to treat or prevent a BM disease or disorder. In such cases, a transgene encoding the BM component of interest, such as collagen type IV, α3, α4, or α5 chain (COL4A3, COL4A4, or COL4A5), is introduced into the cells, which are then infused into a patient such that the cell type(s) affected by the disease is repopulated by the modified cells or tissue following infusion into the subject. The cells or tissue can also be genetically modified using gene therapy techniques known to one skilled in the art (see below) to express a desired gene (e.g., a gene encoding a functional BM component). The modified cells or tissue can then be transplanted into a patient for the treatment or prevention of the BM disease or disorder by any method known in the art that is appropriate for the type of cells or tissue being transplanted and the transplant site.

The cells used in the methods of the invention can be made recombinant using gene therapy techniques. In its broadest sense, gene therapy refers to therapy performed by the administration of a nucleic acid to a subject. The nucleic acid, either directly or indirectly via its encoded protein, mediates a therapeutic effect in the subject. The present invention envisions methods of gene therapy in which a nucleic acid encoding a BM protein is introduced into one or more cells, which are to be administered to a patient suffering from a BM disease or disorder, such that the nucleic acid is expressible by the cells and/or their progeny.

The cells of the present invention can be used in any of the methods for gene therapy available in the art. Thus, the nucleic acid introduced into the cells may encode any desired BM protein, e.g., a BM protein missing or dysfunctional in a BM disease or disorder. For general reviews of the methods of gene therapy, see Goldspiel et al., Clinical Pharmacy 12:488-505, 1993; Wu and Wu, Biotherapy 3:87-95, 1991; Tolstoshev, Ann. Rev. Pharmacol. Toxicol. 32:573-596, 1993; Mulligan, Science 260:926-932, 1993; and Morgan and Anderson, Ann. Rev. Biochem. 62:191-217, 1993. Methods commonly known in the art of recombinant DNA technology that can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

One common method of practicing gene therapy uses viral vectors, for example retroviral vectors (see Miller et al., 1993, Meth. Enzymol. 217:581-599; Boesen et al., Biotherapy 6:291-302, 1994; Clowes et al., J. Clin. Invest. 93:644-651, 1994; Kiem et al., Blood 83:1467-1473, 1994; Salmons and Gunzberg, Human Gene Therapy 4:129-141, 1993; and Grossman and Wilson, Curr. Opin. in Genetics and Devel. 3:110-114, 1993), adenovirus vectors (Kozarsky and Wilson, Current Opinion in Genetics and Development 3:499-503, 1993; Rosenfeld et al., Science ˜52:431-434, 1991; Rosenfeld et al., Cell 68:143-155, 1992; and Mastrangeli et al., J. Clin. Invest. 91:225-234, 1993), adenovirus-associated vectors (AAV; see, for example, Walsh et al. Proc. Soc. Exp. Biol. Med. 204:289-300, 1993), herpes virus vectors, pox virus vectors; non-viral vectors, for example, naked DNA delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, or pressure-mediated gene delivery. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac, Ther. 29:69-92) and may be used in accordance with the present invention, provided that the necessary developmental and physiological functions of the recipient cells are not disrupted. Usually, the method of transfer includes the transfer of a selectable marker to the cells. The cells are then placed under selection to isolate those cells that have taken up and are expressing the transferred gene. Those cells are then delivered to a patient to treat or prevent a BM disease or disorder. The technique should provide for the stable transfer of the gene to the cell, so that the gene is expressible by the cell and preferably heritable and expressible by its cell progeny.

A desired gene can also be introduced intracellularly and incorporated within host precursor cell DNA for expression, by homologous recombination (Koller and Smithies, Proc. Natl. Acad. Sci. USA 86:8932-8935, 1989; Zijlstra et al., Nature 342:435-438, 1989).

Various reports have been presented regarding the efficacy of gene therapy for the treatment of monogeneic diseases, early stage tumors, and cardiovascular disease. (See, e.g., Blaese et al., Science 270:475-480, 1995; Wingo et al., Cancer 82:1197-1207, 1998; Dzao, Keystone Symposium Molecular and Cellular Biology of Gene Therapy, Keystone, Co. Jan. 19-25, 1998; and Isner, Keystone Symposium Molecular and Cellular Biology of Gene Therapy, Keystone, Co. Jan. 19-25, 1998.)

The methods of the invention generally result in a measurable improvement in BM formation and function, e.g., measurable recovery of kidney function due to improvement in GBM. The following results confirm the success of the methods of the invention using an accepted mouse model of human Alport's syndrome (i.e., COL4A3^{-/-} mice; Cosgrove et al., 1996; Hudson, 2004; Hudson et al., 1993; Kalluri et al., 1997; Lemmink et al., 1994; Mochizuki et al., 1994). COL4A3^{-/-} mice treated according to the methods of the invention demonstrate clear improvement in renal function, which is shown by reduced or near normal levels of proteinuria and repair of glomerular architecture, in particular GBM structure.

The invention is further illustrated by the following examples which should not be construed as limiting.

### Treatment Using Bone Marrow Transplantation

Proteinuria

The data illustrate the natural course of progression of proteinuria in COL4A3^{-/-} mice and the beneficial effect of all three modalities of treatment. Proteinuria estimated as urine albumin/creatinine ratio already rose at 8 weeks of age and continued to increase through their death, which occurred at about an age of 20-24 weeks-old.

Treatment of COL4A3^{-/-} mice with bone marrow transplantation from wild type mice reduced the level of proteinuria significantly at 12 weeks- and 22 weeks-old. When we injected 1×10⁶ stem cells into COL4A3^{-/-} mice, proteinuria was diminished to the similar level to bone marrow transplantation. Even repeated blood transfusion from wild type mice, again decreased the level of proteinuria at 12 weeks-old although the decrease is not statistically significant.

Glomerulosclerosis

The COL4A3^{-/-} mouse is characterized by initial glomerulosclerosis, leading to progressive tubulointerstitial disease and ultimately to renal fibrosis at the age of 20-24 weeks. At 22 weeks old, COL4A3^{-/-} mice had fibrous crescents in glomeruli and the glomerular capillary were collapsed. We sacrificed COL4A3^{-/-} mice at 8, 12 and 21 weeks old to see the progression of this disease.

We transplanted 8 weeks old COL4A3^{-/-} mice with bone marrow cells from wild type C57BL/6 mice. Bone marrow transplantation from wild type mice ameliorated the glomerular damage, resulting in an increased percentage of normal glomeruli observed. COL4A3^{-/-} mice showed tubular atrophy and interstitial fibrosis. Treatment with bone marrow transplantation from wild type mice delayed the progression of chronic renal disease, tubular atrophy and interstitial fibrosis.

### Treatment Using Embryonic Stem Cell Injection

Similar beneficial effects were observed when COL4A3^{-/-} mice were treated with intravenous ES cell injection. ES cell injection improved the glomerular structure and slowed the progression of tubular atrophy and interstitial fibrosis.

### Treatment Using Peripheral Blood Transfusion

We also observed that the administration of peripheral blood from wild type mice to COL4A3^{-/-} mice at the age of eight weeks old reproduces the same favorable effect on glomerular architecture and inhibited tubular atrophy and interstitial fibrosis as was seen when bone marrow and ES cell injection was used.

### Transmission Electron Microscopy

Electron microscopy was used to examine kidney tissue at 12 and 22 weeks old to examine ultrastructure changes that occur with disease progression (FIG. 3G). The glomeruli of COL4A3^{-/-} mice observed by transmission electron microscopy showed splitting, lamination and thinning of the glomerular capillary basement membrane. Podocytes show foot process effacement and microvillous transformation, suggesting that COL4A3^{-/-} mice have proteinuria.

At high magnification glomerular basement membrane (GBM) from wild type mice clearly shows defined lamina densa flanked by the lamina rara layers of the basement membrane. The foot process of the glomerular epithelial cells (podocytes) have the characteristic interdigitating appearance. The foot process was connected by a slit diaphragm.

FIG. 3G shows a capillary wall from COL4A3^{-/-} mouse with thinning, multilamination, splitting of the GBM and obliteration of the foot processes or foot process effacement.

Bone marrow transplantation from wild type mice recovered the structure of GBM and interdigitation of foot processes in COL4A3^{-/-} mice.

### Scanning Electron Microscopy

Podocyte surface architecture is shown in FIGS. 3A-3F. In wild type mice, the foot processes interdigitate between two adjacent podocytes to form fern-leaf structures. In COL4A3^{-/-} mice, the characteristic fem-leaf structures disappeared and formed sheet-like architecture. Bone marrow transplantation from wild type mice protected this interdigitation of foot process from becoming sheet-like architecture. The beneficial effect on podocyte interdigitation can be reproduced by the other two modalities of treatment, i.e. stem cell injection and repeated peripheral blood transfusion from wild type mice.

### Beta-Galactosidase and GFP Staining

COL4A3^{-/-} mice exhibit various degrees of damage to podocytes in glomeruli since the age of 8 weeks old. We set out to find whether bone marrow cells derived from Lac-Z- or GFP-expressing mice would be recruited into the damaged glomeruli upon administration to COL4A3^{-/-} mice. We stained the renal tissue from COL4A3^{-/-} mice with an antibody to beta-galactosidase or GFP. As shown in FIG. 4, there were some beta-galactosidase positive cells in the glomeruli of COL4A3^{-/-} mice, whereas no beta-galactosidase positive cells were detected in wild type mice transplanted with bone marrow from Rosa-26 mice. To identity the identity of the beta-galactosidase positive cells found in the glomeruli of COL4A3^{-/-} mice, we double-stained the kidney tissue with synaptopodin (a podocyte marker), CD31 (endothelial cells) or α-1 integrin (mesangial cells). The majority of the β-galactosidase positive cells were identified as podocytes, while the remainder were identified as mesangial cells.

Similar results were obtained using a GFP antibody to stain kidney tissues from COL4A3^{-/-} mice transplanted with bone marrow from GFP-expressing mice or transfused with peripheral blood from GFP mice and further injected with GFP positive embryonic stem cells.

### In Situ Hybridization

In wild type mice, glomerular capillary basement membrane are composed of triple helix of α3, α4 and α5 chain of type IV collagen. These α3, α4 and α5 isoforms are thought to be produced by podocytes. We performed in situ hybridization for type IV collagen α3 chain and found the following.

The mRNA of type IV collagen α3 chain was expressed in podocytes in glomeruli from wild type mice. There was no expression type IV collagen α3 chain mRNA in COL4A3^{-/-} mice. Bone marrow transplantation from wild type mice induced type IV collagen α3 chain expression in newly recruited podocytes in the glomeruli, which promoted type IV collagen α3 chain mRNA expression in glomeruli and the formation of functional BM.

### Immunofluorescence

In the wild type mice ⊕1 and α2 chain isoforms of type IV collagen preferentially localized to the mesangial matrix and α3, α4 and α5 chains were limited to the glomerular capillary wall.

Loss of α3 chain isoforms of type IV collagen in GBM leads to a transition of α1 and α2 chain of type IV collagen from the mesangial GBM (normal distribution) to the capillary GBM (abnormal localization). As expected, in COL4A3^{-/-} mice, α3 chain of type IV collagen is absent in the GBM as well as α5 chain, except for a segmental trace expression of α5 chain.

After the bone marrow transplantation from wild type mice, COL4A3^{-/-} mice showed stippled or segmentally linear staining pattern of α3 chain and almost complete recovery of α5 chain on the GBM. Although α3 and α5 chain were recovered, α1 and α2 chain still existed in the capillary GBM. This is probably because newly formed GBM structure with α1, α2, α3, α4, and α5 chains is not matured completely and preexisting α1 and α2 isoforms support the newly forming GBM.

Similar recovered expression of α3 and α5 chain in the capillary GBM was obtained when COL4A3^{-/-} mice were treated with stem cell injection or multiple repeated transfusion of peripheral blood from wild type mice.

### Discussion

Our results demonstrate the successful treatment of COL4A3^{-/-} mice, a mouse model of human Alport's syndrome (Cosgrove et al., 1996; Hudson, 2004; Hudson et al., 1993; Kalluri et al., 1997; Lemmink et al., 1994; Mochizuki et al., 1994), with progenitor mesenchymal and endothelial COL4A3^{+/+} stem cells leading to a clear improvement in renal function, which is shown by reduced or near normal levels of proteinuria (FIG. 2A), and a lucid amelioration of glomerular architecture, in particular GBM structure (FIG. 4). After treatment with either Lac-Z- or GFP-expressing “stem cells,” clear glomerular localization of Lac-Z or GFP expression could be found, indicating the recruitment of these cells into damaged glomeruli. After recruitment, stem cells subsequently differentiate into podocytes and are able to repair GBM structure, which leads to a marked improvement in renal function.

Moreover, (partial) re-expression of type IV collagen α3 chain in treated COL4A3^{-/-} mice, as revealed by immunofluorescence (FIG. 4), in situ hybridization (FIGS. 5-7) and RT-PCR (FIG. 2B) analysis, leads to restoration of α4 and α5 chain expression. Restoration of type IV collagen α3 chain results in appropriate type IV collagen protomer formation and improved GBM structure, as compared to untreated COL4A3^{-/-} mice. Furthermore, our results demonstrate that stem cells for therapy can be derived from bone marrow, embryonic stem cell culture, or from the circulating stem cell pool of whole peripheral blood, respectively (Lagasse et al., 2000; Roufosse et al., 2004). Blood transfusion is a common, safe, and inexpensive procedure which is performed thousands of times worldwide every day, so the finding that repetitive blood transfusion promotes significant improvement of renal structure and function in COL4A3^{-/-} mice reveals that this method provides an easy and practicable therapy in patients with renal failure due to inherited Alport's syndrome.

In our experimental approach, lesions, in particular damaged GBM due to inherited loss of type IV collagen α3 chain, are already present, and stem cell therapy via bone marrow therapy, ES cell injection, or blood transfusion is performed to treat these pre-existing lesions. Alternatively, these therapies can be provided to patients who do not exhibit pre-existing lesions, but who are likely to develop such lesions due to, e.g., disease or injury, or where the patient has reduced type IV collagen α3 chain expression.

Collectively we have demonstrated in three different and independent approaches, that administrating progenitor mesenchymal and endothelial COL4A3^{+/+} stem cells is results in the recruitment and engraftment of COL4A3^{+/+} stem cells to the damaged GBM of COL4A3^{-/-} mice, which restores (at least partially) the expression of type IV collagen α3 chain and results in improved GBM structure and renal function, respectively. This therapy can be provided to patients with Alport's syndrome to heal their inherited renal failure, thus freeing those patients from lifelong dependency on hemodialysis or the need to undergo repeated kidney transplantation. The most striking finding is that the treatment can be performed in an easy and practicable way by (repeated) blood transfusion.

### Materials and Methods

### Mice

Transgenic mice constitutively expressing lac Z in all tissues (TgR(ROSA26)26Sor), Green Fluorescent Protein (GFP) expressing mice (Tg(ACTbEGFP)1Osb), and wild type C57BL/6 mice were obtained from The Jackson Laboratories (Bar Harbor, Me.: http://www.jax.org) Type IV collagen α3 chain knockout (COL4A3^{-/-}) mice were kindly provided by Dr. Dominic Cosgrove (Omaha, Nebr.) {Cosgrove, 1996 #99} and Dr. Jeffrey Miner (St. Louis, Mo.) {Miner, 1996 #94}, respectively, and back crossed into C57BL/6 mice (The Jackson Laboratories, Bar Harbor, Me.). Mice were maintained at the Beth Israel Deaconess Medical Center animal facility under standard conditions, and fed standard mouse chow and water ad libitum. Homozygous deletion of COL4A3 was confirmed by PCR as described previously {Miner, 1996 #188; Cosgrove, 1996 #145}.

All animal studies were reviewed and approved by the animal care and use committee of Beth Israel Deaconess Medical Center.

### Bone Marrow Transplantation (BMT)

Eight weeks old mice (COL4A3^{-/-}; COL4A3^{+/-}; COL4A3^{+/+}) were lethally irradiated with 10 Gy of a ¹³⁷Cesium gamma-source. Mice were rescued by intravenous administration of 2-5×10⁶ unfractionated BM cells 24 hours after irradiation. BM cells were obtained aseptically by flushing femurs, tibias and humeri of ROSA 26, GFP, or COL4A3^{-/-} donor mice, respectively. After BM transplantation mice were housed in autoclaved cages and received antibiotics for three weeks with drinking water (0.1% Amoxicillin (Sigma, St Louis, Mo.) and 0.015% Enrofloxacine (FLUKA-Sigma, St. Louis, Mo.). To study the possible influence of bone marrow transplantation procedure itself, in particular to exclude an anti-inflammatory effect of the irradiation process on the progression of the renal disease, COL4A3^{-/-} bone marrow was transplanted into COL4A3^{-/-} mice and COL4A3^{+/+} wild type mice, respectively.

### Proteinuria

Urinary albumin and creatinine concentration were estimated using a colorimetric assay according to the manufacturer's recommendation (Sigma St Louis, Mo.). Urine albumin excretion was estimated as the quotient of urine albumin and urine creatinine as previously described (Sugimoto, 2003).

### Immunohistochemistry

Immunofluorescence staining was performed as described previously (Cosgrove, 1996; Sugimoto, 1998; Mundel, 1997; Mundel, 1999) with some modification. Animals were side-separately, transcardially perfused with 2% PBS buffered formalin before organs were harvested. For α3 and α5 chain of type IV collagen staining 4 μm cryosections were fixed in 100% ethanol for 5 min at −20° C., rinsed in PBS, and treated with 0.1 M glycine-6 M Urea (pH 3.5) for 1 h at 4° C. Primary antibodies to α3 and α5 chain of type IV collagen were applied for 1 h at room temperature and sections were reacted with FITC-conjugated anti-rabbit IgG (Jackson ImmunoResearch, West Grove, Pa.) for 1 h at room temperature. Sections were then incubated with an antibody against α1 and α2 chain of type IV collagen (Southern Biotechnology Associates, Inc, Birmingham, Ala.) for 1 h at room temperature, followed by incubation with rhodamine-conjugated anti-goat IgG (Jackson ImmunoResearch). A similar method was employed for immunostaining of β-galactosidase, α1-integrin, CD31, and entactin without urea treatment of glass slides.

For GFP staining, 10% formalin-fixed paraffin embedded tissue sections were used. After deparaffinization glass slides were treated in a microwave oven for 5 min and FITC-conjugated goat anti-GFP antibody (GeneTex, abcam, Cambridge, Mass.) was applied for 1 h at room temperature, followed by 1 h incubation with mouse anti-synaptopodin (a generous gift from Dr. Peter Mundel, New York, N.Y.) at room temperature followed by rhodamine-conjugated anti-mouse IgG for 1 h at room temperature.

### Histological Assessment of Renal Injury

Kidney tissues were fixed in 4% paraformaldehyde and embedded in paraffin, cut at 4 μm and stained with hematoxylin-eosin (HE), periodic acid Shiff (PAS) and Masson trichrome. The extent of renal injury was assessed by morphometry of the glomerular disease, tubular atrophy and interstitial fibrosis as previously described (Zeisberg, 2003). The relative interstitial volume was evaluated using a 10-mm² graticule fitted into the microscope. Five randomly selected cortical areas, which included glomeruli, were evaluated for each animal. Tubules were evaluated for their lumen and thickened basement membranes to estimate percentage of atrophic tubules.

Photomicrographs were taken using a Zeiss Axioskop 2plus fluorescence microscope and Axiovion digital imaging software (Carl Zeiss MicroImaging, Inc., Thornwood, N.Y.).

### Electron Microscopy

Transmission electron microscopy (TEM) and scanning electron microscopy (SEM) were performed as previously described (Cosgrove, 2000; Mundel, 1999; and Mundel, 1997).

For scanning electron microscopy, small pieces (approximately 2 mm cubes) of kidney cortex were fixed in 3% phosphate-buffered glutaraldehyde, then postfixed in 1% phosphate-buffered sodium tetroxide. Samples were then dehydrated in graded ethanol, and critical point-dried in carbon dioxide. The cubes were then cracked in pieces by stressing them with the edge of a razor blade, and mounted with glue onto stubs with cracked surface facing upward. The surface was sputter-coated using gold/palladium and visualized with a scanning electron microscope.

### In Situ Hybridization

In situ hybridization was performed as previously described (Sayers, 1999).

### Blood Transfusion Experiments

GFP transgenic mice were used as peripheral blood donors. One milliliter whole blood was collected under isoflurane anesthetic from the retroorbital venous plexus from each donor mouse. Blood was collected into heparin-supplemented Eppendorf tubes using microcapillary pipettes, and was kept at room temperature until transfusion. Eight weeks-old COL4A3^{-/-} mice were anesthetized by isoflurane inhalation and 150-400 μl whole donor blood were injected into the retroorbital venous plexus using 20 gauge needle syringes. Blood transfusion was performed twice a week for three weeks. Transfused mice were sacrificed at 12.5 weeks of age, and tissues were collected for further analysis.

### Cell Culture of Embryonic Stem Cells

Murine undifferentiated embryonic stem cells (ES cells) expressing green fluorescent protein (GFP) were a generous gift from Dr. George Q. Daley, Children's Hospital, Boston. Undifferentiated ES cells were cultured on mitotically inactivated primary mouse embryonic fibroblasts in Dulbecco's Modified Eagle Medium (DMEM, Gibco), supplemented with 15% fetal bovine serum (Gibco), 1 M HEPES buffer (Sigma, St Louis, Mo.), 100 mM sodium pyruvate (Sigma), 0.12% monothioglycerol (Sigma), and 1000 IU/ml recombinant leukemia inhibitory factor (LIF; Chemicon International, Temecula, Calif.).

On the day of stem cell transplantation, feeder cells were removed by incubation of the cell suspension for 30 min at 37° C. and then resuspended in PBS; the procedure was repeated once.

### Stem Cell Transplantation

Twelve weeks old COL4A3^{-/-} mice were anesthetized by isoflurane inhalation and 1×10⁶ ES cells were injected through the retroorbital venous plexus using 20 gauge needle syringes. Stem cell transplanted mice were housed in the institutional animal facility and given standard chow and water ad libitum. Mice were sacrificed at 20 weeks of age and kidney tissues were collected for histological analysis.

### RT-PCR

Total RNA was isolated from snap frozen kidneys using Trizol reagent (Invitrogen) in accordance with the manufacturer's instructions. RT-PCR was performed using SuperScript III One-Step RT-PCR System (Invitrogen) in accordance with the manufacturer's instructions, Primers used including GAPDH,

### Statistical Analysis

All values are expressed as mean±SE. Analysis of variance (ANOVA) was used to determine statistical differences among more than two groups. Further analysis was carried out using Student's t-test with Fisher's correction to identify significant differences. P<0.05 was considered statistically significant.

### Other Embodiments

The contents of all references, patents, and published patent applications cited throughout this application are hereby incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

What is claimed is:

1. A method for treating or preventing a basement membrane disease or disorder comprising administering to a patient in need thereof a stem cell from bone marrow, peripheral blood, or umbilical cord blood of a subject lacking said basement membrane disease or disorder, wherein said administering treats or prevents said basement membrane disorder.

2. The method of claim 1, comprising, prior to administering said stem cell, performing a diagnostic test on said patient to determine that said patient has said basement membrane disorder.

3. The method of claim 1, wherein said disease or disorder is selected from Alport's syndrome, Knoblach syndrome, hematuria, epidermolysis bullosa, thin basement membrane nephropathy, diabetic nephropathy, hereditary nephritis, or any disease with defects in basement membranes and extracellular matrix.

4. The method of claim 1, wherein said stem cell is from bone marrow.

5. The method of claim 1, wherein said stem cell is from peripheral blood.

6. The method of claim 5, wherein said peripheral blood is mobilized.

7. The method of claim 1, wherein said stem cell is from umbilical cord blood.

8. The method of claim 1, wherein said stem cell is a mesenchymal stem cell.

9. The method of claim 1, wherein said stem cell is an endothelial stem cell.

10. The method of claim 1, wherein said basement membrane disease or disorder is characterized by a defect in one or more basement membrane components.

11. The method of claim 10, wherein said basement membrane component is selected from collagen, laminin, a heparan sulfate proteoglycan, entactin/nidogen, agrin, SPARC/BM-40, osteopontin, and a fibulin.

12. The method of claim 11, wherein said collagen is selected from type IV collagen.

13. The method of claim 12, wherein said basement membrane component is the α3, α4, or α5 chain of type IV collagen.

14. The method of claim 13, wherein said basement membrane component is the α3 chain of type IV collagen.

15. The method of claim 1, wherein said administering comprises injection into the blood stream of said patient.

16. The method of claim 1, wherein said stem cell is present in a composition.

17-30. (canceled)

31. A method for treating or preventing a basement membrane disease or disorder comprising administering to a patient a vector comprising a functional basement membrane component, wherein said administering treats or prevents said basement membrane disorder.

32. The method of claim 31, wherein said vector is a viral vector comprising a transgene in an expressible genetic construct, wherein said transgene expresses said basement membrane component.

33. The method of claim 32, wherein said viral vector is an adenoviral vector.

34. The method of claim 31, wherein said vector is a cell that has been modified to express said basement membrane component.

35. The method of claim 34, wherein said cell is an autologous or an allogeneic cell.

36. The method of claim 34, wherein said cell has been modified by infection with a viral vector.

37. The method of claim 36, wherein said viral vector is an adenoviral vector.

38. The method of claim 31, wherein said patient is a human.

39. The method of claim 31, comprising, prior to administering said vector, performing a diagnostic test on said patient to determine that said patient has said basement membrane disorder.

40. The method of claim 31, wherein said disease or disorder is selected from Alport's syndrome, Knoblach syndrome, hematuria, epidermolysis bullosa, thin basement membrane nephropathy, diabetic nephropathy, hereditary nephritis, or any disease with defects in basement membranes and extracellular matrix.

41. The method of claim 34, wherein said cell is a stem cell.

42. The method of claim 41, wherein said stem cell is from bone marrow.

43. The method of claim 41, wherein said stem cell is from peripheral blood.

44. The method of claim 43, wherein said peripheral blood is mobilized.

45. The method of claim 41, wherein said stem cell is from umbilical cord blood.

46. The method of claim 34, wherein said stem cell is a mesenchymal stem cell.

47. The method of claim 34, wherein said stem cell is an endothelial stem cell.

48. The method of claim 31, wherein said basement membrane disease or disorder is characterized by a defect in said basement membrane component.

49. The method of claim 48, wherein said basement membrane component is selected from collagen, laminin, a heparan sulfate proteoglycan, entactin/nidogen, agrin, SPARC/BM-40, osteopontin, and a fibulin.

50. The method of claim 49, wherein said collagen is selected from type IV collagen.

51. The method of claim 50, wherein said basement membrane component is the α3, α4, or α5 chain of type IV collagen.

52. The method of claim 51, wherein said basement membrane component is the α3 chain of type IV collagen.

53. The method of claim 31, wherein said administering comprises injection of said vector into the blood stream of said patient.
